(19) United States
(12) Patent Application Publication
(10) Pub. No.: US 2022/0105214 A1
(43) Pub. Date: Apr. 7, 2022

(54) DISINFECTION PASSAGEWAY

(71) Applicant: Rajesh PATEL, Lewisburg, WV (US)

(72) Inventor: Rajesh PATEL, Lewisburg, WV (US)

(21) Appl. No.: 17/060,687

(22) Filed: Oct. 1, 2020

(51) Int. Cl. A61L 2/18

(52) U.S. Cl. CPC A61L 2/18; A61L 2202/20; A61L 2202/15; A61L 2202/122

(57) ABSTRACT

The present invention relates to a disinfection passageway with an alerting system. The passageway includes: a first sidewall; a second sidewall; and a top cover extending between the first sidewall and the second sidewall. The passageway also has a storage tank attached to the outer surface of the first sidewall. The storage tank stores sanitizing liquid for dispensing through a plurality of nozzles on the inner surface of at least one sidewall. A temperature sensor is also attached to the passageway, wherein the temperature sensor is adapted for measuring a body temperature of a user. Finally, a hand sanitizer dispenser on the inner surface of at least one side wall adapted for dispensing hand sanitizer.

### BACKGROUND OF THE INVENTION

### Field of Invention

The present invention relates to a disinfection passageway to assist in providing disinfecting options for a user.

### Description of Related Art

In the present society a high concern and emphasis has been placed on controlling the spread of viruses and bacteria especially due to the ongoing pandemic that has engulfed the world. The use of disinfectants and face masks has increased tremendously to reduce the spread of viruses and bacteria. Many individuals are highly concerned and have increased using sprays to clean surfaces, chairs, handles, doors, doorways, seats, tables and trays that are commonly used in everyday practices. It has become fairly important that individuals are using their face masks to cover their face, in particular their mouth and nose areas to avoid the transmission of bacteria and viruses.

Individuals are further going to lengths to protect their hands through the use of gloves and through the use of hand wipes that are used commonly to wipe hands after using the bathroom or in certain instances just simply to remove any bacteria from the skin after engaging in public activities. Many businesses are requiring individuals, and further, governments are passing laws, that require individuals to wear face masks upon entering a public place. These procedures are being coordinated to attempt to limit the spread of viruses and bacteria. As a result, it would be highly advantageous for the general public and businesses to implement further safety features to diminish and prevent the spread of viruses and bacteria.

### BRIEF DESCRIPTION OF DRAWINGS

FIG. 1 depicts a disinfection passageway according to the present invention.

FIG. 2 depicts a side-view of the disinfection passageway according to the present invention.

FIG. 3 depicts a perspective view of the disinfection passageway according to the present invention.

### DETAILED DESCRIPTION

The present invention relates to a disinfection passageway and a method of disinfecting a user who passes there through. The present invention provides a passageway that includes particular hardware functions for disinfecting purposes. A temperature sensor is provided on one sidewall of the passageway that senses the body temperature of a user before entering into the passageway. A second feature is a hand sanitizing dispenser that is provided on the other sidewall of the passageway. A third feature is misting that takes place once an individual enters the passageway. These three disinfecting and testing aspects are signaled and alerted through an alerting light that is placed on the top of the passageway. The alerting light signals when an individual has engaged with the temperature sensor to determine body temperature, secondly it signals when an individual engages the sanitizing dispenser and finally, once the sanitizing dispenser is used a mist is dispersed over the individual while within the passageway and a third signaling light is activated.

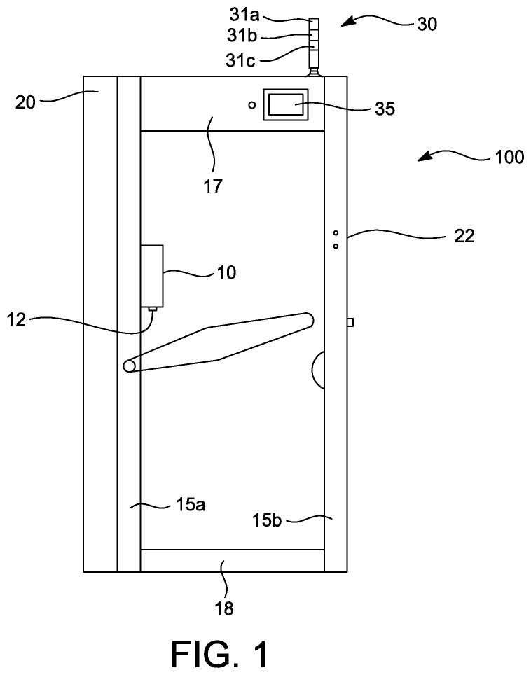

In reference to FIG. 1, a Passageway 100 is depicted. The Passageway 100 includes a Base/Threshold 18, where a first Sidewall 15a and a second Sidewall 15b extend therefrom. A Top Cover 17 encloses the Passageway 100 and connects with Sidewalls 15a, 15b. A temperature sensor 22 is provided on the Sidewall 15b. The temperature sensor 22 requires the user to come in close proximity thereof to determine body temperature. Once the body temperature is taken by the Sensor 22 and the user's temperature is shown to be appropriate or within the normal human body temperature of 98.6, Alerting Light 30 provides a First Signal 31c as shown on the top of the Top Cover 17.

The Alerting Light 30 is further used in conjunction with the disinfecting aspects of the Passageway 100. A hand sanitizer dispenser 10 is provided on the inside of Sidewall 15a that releases hand sanitizer through an opening 12 as shown in FIG. 1. Further, a LED Display 35 is shown on the Top Cover 17. Extending from the Sidewall 15a is a Tank 20. The Tank 20 includes a disinfectant liquid that is dispensed through a plurality of Nozzles 25 on the inside of Sidewalls 15a, 15b. Further, Nozzles 25 may be placed on the inner surface of the Top Cover 17 to further dispense disinfecting spray. This light mist of disinfectant covers the user once the user enters the passageway. In one particular embodiment, the Base/Threshold 18 may include heating elements to dry any excess moisture accumulating over the Base/Threshold 18

The Alerting Light 30 includes the First Indicator 31a that is used in conjunction with the Temperature Sensor 22 and a Second Indicator 31b is used in conjunction with the hand sanitizer dispenser 10 and a Third Indicator 31c alerts once the mist has been dispensed over the user.

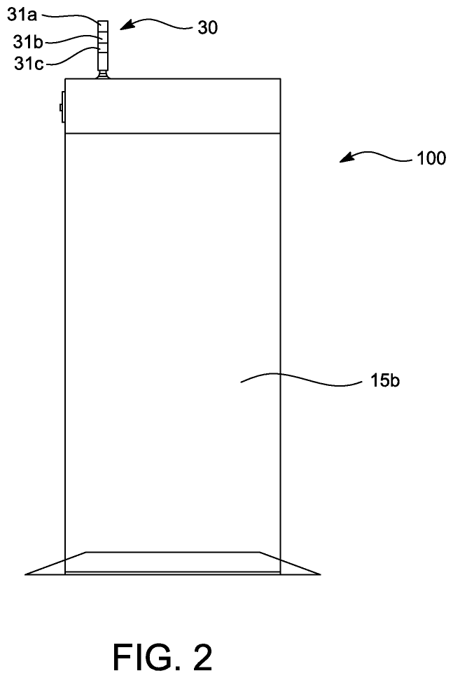

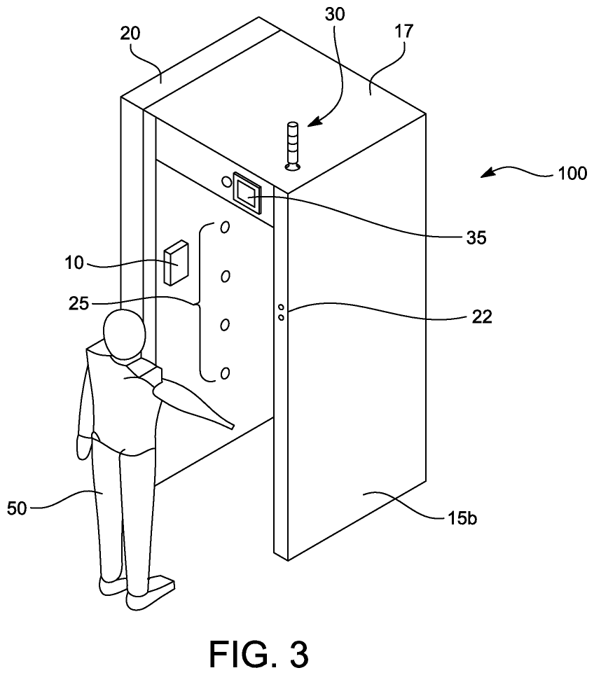

Further aspects of the Passageway 100 are shown in FIGS. 2 and 3. The side view, FIG. 2, shows the outer surface of Sidewall 15b and the Alerting Light 30 extending from the Top Cover 17. The perspective view shown in FIG. 3 shows the user just outside Passageway 100. This User 50 first approaches the Body Temperature Check Sensor 22 to activate a safe body temperature before entry therein. Next, the user 50 must engage the hand sanitizer dispensed from Dispenser 10 and finally once the user is within the passageway, the aerial disinfectant is dispensed over the User 50.

The storage tank 20 for the aerial disinfecting solution can vary in size based upon customer needs and may include connections for refilling the tank with water and sanitizer as needed. The Base 18 may include a surface heater to prevent the build-up of fluid on the Base 18 as the user approaches through the Passageway 100. The passageway may require manual cleaning of the Base or Threshold 18 to ensure the surface is dry for users to disburse over thereof.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A disinfection passageway comprising:
a. a first sidewall;
b. a second sidewall;
c. a top cover extending between the first side wall and the second sidewall;
d. a storage tank attached to an outer surface of the first side wall, wherein the storage tank includes liquid for dispensing through a plurality of nozzles on the inner surface of at least one sidewall;
e. a temperature sensor on the second sidewall, wherein the temperature sensor is adapted for measuring a body temperature of a user; and
f. a hand sanitizer dispenser on the inner surface of at least one side wall.

2. The disinfection passageway according to claim 1, further including an alerting system on top of the top cover.

3. The disinfection passageway according to claim 2, where the alerting system includes a plurality of lights.

4. The disinfection passageway according to claim 3, where the plurality of lights includes a first light, a second light and a third light.

5. The disinfection passageway according to claim 4, wherein the first light signals compliance with use of temperature sensor; the second light signals compliance with use of the hand sanitizer dispenser and the third light signals completed application of a disinfection solution.

6. The disinfection passageway according to claim 1, wherein the storage tank includes a disinfection solution.

7. A method of disinfecting a user passing through a passageway comprising the steps of:
a. testing a body temperature of a user;
b. signaling completion of the step of testing;
c. dispensing a hand sanitizer to the user;
d. signaling completion the step of dispensing;
e. emitting a misting spray over the user; and
f. signaling completion of the step of emitting.

8. The method according to claim 7, where steps of signaling are accomplished through a lighting system.

9. The method according to claim 7, where step of emitting is accomplished through a plurality of nozzles.

10. The method according to claim 7, where the step of testing is accomplished through a temperature sensor.
